(19) United States
(12) Patent Application Publication
(10) Pub. No.: US 2018/0334664 A1
(43) Pub. Date: Nov. 22, 2018

(54) REGENERATION OF CORPUS CAVERNOSAL TISSUE

(71) Applicant: Creative Medical Technologies, Inc., Phoenix, AZ (US)

(72) Inventors: Thomas Ichim, San Diego, CA (US); Annette Marleau, San Diego, CA (US)

(73) Assignee: Creative Medical Technologies, Inc., Phoenix, AZ (US)

(21) Appl. No.: 15/981,726

(22) Filed: May 16, 2018

Related U.S. Application Data
(60) Provisional application No. 62/506,886, filed on May 16, 2017.

(51) Int. Cl. C12N 13/00; C12N 5/077; C12N 5/0775; A61K 35/28; A61P 15/12

(52) U.S. Cl. CPC C12N 13/00; C12N 5/0669; A61P 15/12; C12N 5/0665; A61K 35/28; C12N 5/0653

(57) ABSTRACT

Disclosed are methods, devices, and techniques useful for enhancing function of an organ or cellular graft through administration of light frequencies sufficient to enhance desired therapeutic activity. In one embodiment a bone marrow cellular population is treated with one or more wavelengths of low level laser irradiation at a sufficient energy to enhance engraftment, trophic, and regenerative activity in the corpus cavernosum. In another embodiment the recipient penile body is treated with one or more wavelengths of low level laser irradiation at a sufficient energy to enhance chemoattraction and growth factor secretion.

### CROSS-REFERENCE TO RELATED APPLICATIONS

This application claims benefit of U.S. Provisional Application No. 62/506,886, filed May 16, 2017, which is hereby incorporated by reference in its entirety.

### FIELD OF THE INVENTION

The invention pertains generally to the area of cellular transplantation, more specifically, the invention relates to the need for enhancing engraftment and function of cellular grafts after transplantation. The invention also belongs to the field of photoceuticals and utilization of light therapy for augmentation of graft efficiency.

### BACKGROUND

Cellular transplantation offers the possibility of curing many diseases previously considered uncurable. Perhaps the biggest success of this approach is witnessed in the area of hematopoietic stem cell transplants, which have cured not only patients with leukemias, but also several genetic abnormalities [1]. In situations of hematopoietic stem cell transplantation recipient bone marrow is usually ablated in order to provide “space” for the donor bone marrow cells. Additionally, when treatment of hematological malignancies is being performed, the recipient cells need to be eradicated in order to cure the neoplasia. Unfortunately the process of hematological ablation involves exposing the patient to a period of immune deficiency. During this period multiple infections occur, which in a non-insignificant number of cases are lethal. Accordingly, it is of great interest in the art to accelerate the process of donor bone marrow engraftment and hematopoietic reconstitution. Another cause of stem cell transplant associated mortality is graft versus host disease (GVHD). In this condition the donor stem cell graft contains immune cells that initiate attack on the recipient. Cord blood transplants have significantly less GVHD as compared to bone marrow, however cord blood cells have a delayed engraftment time, thus exposing the patient to elevated risk of infections. Thus there is a need in the art to enhance stem cell engraftment and reconstitution in cells such as cord blood stem cells.

Cellular transplants have been useful in the treatment of type I diabetes. The Edmonton Protocol is an example of islet transplantation that has demonstrated positive results [2]. Unfortunately 2-3 donor pancreases are needed per recipient and long-term graft survival rarely occurs. The efficacy of the Edmonton Protocol could be increased if the problems of poor vascularization, post-implantation apoptosis, and growth factor secretion of the implanted islets could be modified. Improved cell graft quality has been achieved with methods such as co-addition of bone marrow cells [3], treatment with hyperbaric oxygen [4], or addition of growth factors [5]. Co-administration of bone marrow cells with the islet graft may cause potential problems in terms of ectopic tissue growth. Furthermore this procedure has not been performed clinically on a wide-spread basis. Addition of growth factors may trigger subdormant tumors. Therefore there are currently limited methods to increase viability of a cellular graft. This problem is not only apparent in the case of islet transplantation but also for neuronal and hepatocyte transplantation.

### SUMMARY

Embodiments herein are directed to methods of increasing efficacy of a cell for use in transplantation by pretreatment of said cell prior to transplantation with a laser irradiation of at least one wavelength, said wavelength(s) in a range between about 400 nanometers and about 1070 nanometers administered for a total energy of 100 W/cm² to approximately 10 W/cm². Additional embodiments are directed to methods of treating suboptimal function of corpus cavernosal tissue comprising the steps of: a) selecting a patient whose corpus cavernosal tissue is functioning suboptimally; b) providing said patient a mobilizing agent or therapy sufficient to increase circulating regenerative cells; and c) providing said patient a chemoattractant to selectively induce homing of said regenerative cells to said corpus cavernosal tissue.

### BRIEF DESCRIPTION OF THE DRAWINGS

FIG. 1 shows a medical device for pretreating cells before engraftment.

### DESCRIPTION OF THE INVENTION

The invention provides means of “preactivating” a cellular graft before implantation. Said “preactivation” refers to induction of biochemical processes within the graft so as to allow for: a) increased viability; b) augmented function; c) accelerated integration with the tissue in which implantation of cellular graft has occurred. In the context of hematopoietic stem cell grafts the present invention provides methods of augmenting ability of transplanted stem cells to enter and incorporate into the bone marrow niche, to self renew and proliferate once established in said niche, and to generate blood cells.

In one embodiment the invention teaches the use of low level laser irradiation as a means of increasing practivating stem cells before infusion. Wavelengths and energy of low level laser irradiation useful for stimulation of stem cell proliferation may be chosen based on mitogenesis, colony forming unit, cytokine stimulation or SCID-repopulating unit experiments. Stem cell populations include bone marrow, cord blood, or mobilized peripheral blood mononuclear cells, as well as selected CD34 and/or CD133 cells. It is known to one of skill in the art that wavelengths and energies useful for the practice of the invention may be chosen based ability to stimulate proliferation, cytokine secretion or activity of cells similar to hematopoietic stem cells. In one particular embodiment stem cells are treated for approximately 60 seconds with a helium neon (He—Ne) laser at a wavelength of 632.8 nm and at a power of approximately 2.8 mW following guidance provided in previous published work in the area of porcine granulosa cells [6]. Alternatively, the same wavelength may be used at an energy of approximately 1.5 J/cm² which was demonstrated to stimulate cytokine production from cultured keratinocyte cells [7]. Other wavelengths may be used based on guidance provided from publications describing stimulatory activity on non-hematopoietic stem cells. For example, administration of 0.96 J/cm² of energy at 635 nm was shown sufficient to increase mesenchymal stem cells homing to infarct areas [8]. This wavelength and energy is applied in the context of the present invention as a means of pre-activating hematopoietic stem cell prior to implantation. Similar wavelength at 0.96 J/cm² was capable of eliciting cytokine production and augmenting differentiation processes [9], thus could be used within the context of the current invention. Additionally, wavelengths of 810 nm have also been useful in stimulating mesenchymal stem cell homing and activity at energies between 1-3 J/cm [10, 11] and are also contemplated within the current invention for hematopoietic stem cell pre-activation. The cited publications did not have the intention of activating stem cells for use in the particularly unique process of preactivation of hematopoietic stem cells before infusion but rather demonstrate that light-based intervention is capable of eliciting cellular activities. The unique process of the current invention is the new application of the well-known activities of low level lasers to the process of hematopoietic reconstitution.

Within the context of the current invention low level laser irradiation may be used in combination with other agents known to augment various processes of hematopoietic reconstitution. Means of accelerating this process include modification of progenitor cells by alteration of adhesion molecule activity through such manipulations such as fucosylation of cell surface molecules, cytokine pretreatment, exposure to heat, or treatment with epigenetic acting factors such as 5-azacytidine, valproic acid, trichostatin-A, or sodium phenylbutyrate.

In the practice of the invention stem cells useful for hematopoietic reconstitution may be preactivated and subsequently administered together with growth factors. Administration of said growth factors may occur prior to stem cell administration, concurrently with, or subsequently after. Granulocyte colony stimulating factor (G-CSF) is an example of a growth factor used in clinical practice to augment hematopoietic, and particularly granulocytic differentiation. In one embodiment of the current invention G-CSF is used as an adjuvant to accelerate hematopoietic reconstitution of low level laser pretreated hematopoietic stem cells.

One of the major dose-limiting toxicities of chemotherapy is cytopenia. Use of G-CSF to augment neutrophil production after or concurrent with chemotherapy is common-place in oncology clinics. The current invention teaches that administration of low level laser irradiation to the long bones of a hematopoietically-compromised patient induces acceleration of hematopoietic recovery and shortens cytopenic time. Low level irradiation frequency and power must be sufficient to penetrate the long bones and may be chosen between about 400 nanometers to about 1070 nanometers administered for a total energy of 100 .mu.W/cm.sup.2 to approximately 10 W/cm.sup.2. In one particular embodiment a wavelength of approximately 808 nm is used.

Administration of low level laser irradiation to long bones may also be used for acceleration of stem cell reconstitution after administration. In this embodiment wavelengths are chosen based on ability to increase production of the chemokine stromal derived factor (SDF)-1 from the bone marrow stromal cells. Wavelengths useful for this application range from 400 nanometers to about 1070 nanometers administered for a total energy of 100 .mu.W/cm.sup.2 to approximately 10 W/cm.sup.2. One particular approach for ensuring tissue penetration is using similar parameters as reported by the Photothera group in which 808 nm wavelength was sufficient to penetrate the skull bone and elicit effects in patients post-stroke [12]. The current invention teaches that screening for optimized wavelengths may be performed in vitro using mesenchymal stem cells or osteoblasts as target cells and assessing production of SDF-1 using methods such as enzyme linked immunosorbent assay (ELISA) or reverse transcriptase polymerase chain reaction (RT-PCR).

For the practice of the invention, Lasers (Light amplification by stimulated emission of radiation) are devices that typically generate electromagnetic radiation which is relatively uniform in wavelength, phase, and polarization, originally described by Theodore Maiman in 1960 in the form of a ruby laser [13]. These properties have allowed for numerous medical applications including uses in surgery, activation of photodynamic agents, and various ablative therapies in cosmetics that are based on heat/tissue destruction generated by the laser beam [14-16]. These applications of lasers are considered “high energy” because of their intensity, which ranges from about 10-100 Watts. The subject of the current paper will be another type of laser approach called low level lasers (LLL) that elicits effects through non-thermal means. This area of investigation started with the work of Mester et al who in 1967 reported non-thermal effects of lasers on mouse hair growth [17]. In a subsequent study [18], the same group reported acceleration of wound healing and improvement in regenerative ability of muscle fibers post wounding using a 1 J/cm2 ruby laser. Since those early days, numerous in vitro and in vivo studies have been reported demonstrating a wide variety of therapeutic effects involving LLL, a selected sample of which will be discussed below. In order to narrow our focus of discussion, it is important to first begin by establishing the current definition of LLL therapy. According to Posten et al [19], there are several parameters of importance: a) Power output of laser being 10(−3) to 10(−1) Watts; b) Wavelength in the range of 300-10,600 nm; c) Pulse rate from 0, meaning continuous to 5000 Hertz (cycles per second); d) intensity of 10 to 100 W/cm(2) and dose of 0.01 to 100 J/cm(2). Most common methods of administering LLL radiation include lasers such as ruby (694 nm), Ar (488 and 514 nm), HeNe (632.8 nm), Krypton (521, 530, 568, and 647 nm), Ga—Al—As (805 or 650 nm), and Ga—As (904 nm). Perhaps one of the most distinguishing features of LLL therapy as compared to other photoceutical modalities is that effects are mediated not through induction of thermal effects but rather through a process that is still not clearly defined called “photobiostimulation”. It appears that this effect of LLL is not depend on coherence, and therefore allows for use of non-laser light generating devices such as inexpensive Light Emitting Diode (LED) technology [20]. It is the purpose of the current invention to stimulate angiogenic, growth factor producing properties, and penile regenerative properties of autologous bone marrow cells through exposure to photoceuticals prior to treatment of patient.

To date several mechanisms of biological action have been proposed for augmentation of cellular activity using light based approaches. Without being bound to theory, the invention aims to stimulate these properties in bone marrow cells to be used for treatment of patients with erectile dysfunctin, these include augmentation of cellular ATP levels [21], manipulation of inducible nitric oxide synthase (iNOS) activity [22, 23], suppression of inflammatory cytokines such as TNF-alpha, IL-1beta, IL-6 and IL-8 [24-28], upregulation of growth factor production such as PDGF, IGF-1, NGF and FGF-2 [28-31], alteration of mitochondrial membrane potential [21, 32-34] due to chromophores found in the mitochondrial respiratory chain [35, 36] as reviewed in [37], stimulation of protein kinase C (PKC) activation [38], manipulation of NF-kB activation [39], direct bacteriotoxic effect mediated by induction of reactive oxygen species (ROS) [40], modification of extracellular matrix components [41], stimulation of mast cell degranulation [42], upregulation of heat shock proteins [43] and anti apoptotic effects (Frank et al) Unfortunately these effects have been demonstrated using a variety of LLL devices in non-comparable models. To add to confusion, dose-dependency seems to be confined to such a narrow range or does not seem to exist in that numerous systems therapeutic effects disappear with increased dose.

Descriptions of utilizing lasers and other light sources for stimulation of therapeutic activities of stem cells are modified based on existing knowledge in the art, which is reviewed herein. In 1983, one of the first studies to demonstrate in vitro effects of LLL was published. The investigators used a helium neon (HeNe) laser to generate a visible red light at 632.8 nm for treatment of porcine granulosa cells. The paper described upregulation of metabolic and hormone-producing activity of the cells when exposed for 60 seconds to pulsating low power (2.8 mW) irradiation [6]. The possibility of modulating biologically-relevant signaling proteins by LLL was further assessed in a study using an energy dose of 1.5 joules/cm(2) in cultured keratinocytes. Administration of HeNe laser emitted light resulted in upregulated gene expression of IL-1 and IL-8 [7]. Production of various growth factors in vitro suggests the possibility of enhanced cellular mitogenesis and mobility as a result of LLL treatment. Using a diode-based method to generate a similar wavelength to the HeNe laser (363 nm), Mvula et al reported in two papers that irradiation at 5 J/cm(2) of adipose derived mesenchymal stem cells resulted in enhanced proliferation, viability and expression of the adhesion molecule beta-1 integrin as compared to control [44, 45]. In agreement with possible regenerative activity based on activation of stem cells, other studies have used an in vitro injury model to examine possible therapeutic effects. Migration of fibroblasts was demonstrated to be enhanced in a “wound assay” in which cell monolayers are scraped with a pipette tip and amount of time needed to restore the monolayer is used as an indicator of “healing”. The cells exposed to 5 J/cm(2) generated by an HeNe laser migrated rapidly across the wound margin indicating a stimulatory or positive influence of phototherapy. Higher doses (10 and 16 J/cm(2)) caused a decrease in cell viability and proliferation with a significant amount of damage to the cell membrane and DNA [46]. In order to examine whether LLL may positively affect healing under non-optimal conditions that mimic clinical situations treatment of fibroblasts from diabetic animals was performed. It was demonstrated that with the HeNe laser dosage of 5 J/cm(2) fibroblasts exhibited an enhanced migration activity, however at 16 J/cm(2) activity was negated and cellular damage observed [47]. Thus from these studies it appears that energy doses from 1.5 joules/cm2 to 5 joules/cm2 are capable of eliciting “biostimulatory effects” in vitro in the HeNe-based laser for adherent cells that may be useful in regeneration such as fibroblasts and mesenchymal stem cells.

Studies have also been performed in vitro on immunological cells. High intensity HeNe irradiation at 28 and 112 J/cm(2) of human peripheral blood mononuclear cells, a heterogeneous population of T cells, B cells, NK cells, and monocytes has been described to induce chromatin relaxation and to augment proliferative response to the T cell mitogen phytohemagluttin [48]. In PBMC, another group reported in two papers that interleukin-1 alpha (IL-1 alpha), tumor necrosis factor-alpha (TNF-alpha), interleukin-2 (IL-2), and interferon-gamma (IFN-gamma) at a protein and gene level in human peripheral blood mononuclear cells (PBMC) was increased after HeNe irradiation at 18.9 J/cm(2) and decreased with 37.8 J/cm(2) [49, 50]. Stimulation of human PBMC proliferation and murine splenic lymphocytes was also reported with HeNe LLL [51, 52]. In terms of innate immune cells, enhanced phagocytic activity of murine macrophages have been reported with energy densities ranging from 100 to 600 J/cm(2), with an optimal dose of 200 J/cm(2) [53]. Furthermore, LLL has been demonstrated to augment human monocyte killing mycobacterial cells at similar densities, providing a functional correlation [54].

Thus from the selected in vitro studies discussed, it appears that modulation of proliferation and soluble factor production by LLL can be reliably reproduced. However the data may be to some extent contradictory. For example, the over-arching clinical rationale for use of LLL in conditions such as sinusitis [55], arthritis [56, 57], or wound healing [58] is that treatment is associated with anti-inflammatory effects. However the in vitro studies described above suggested LLL stimulates proinflammatory agents such as TNF-alpha or IL-1 [49, 50]. This suggests the in vivo effects of LLL may be very complex, which to some extent should not be surprising. Factors affecting LLL in vivo actions would include degree of energy penetration through the tissue, the various absorption ability of cells in the various tissues, and complext chemical changes that maybe occurring in paracrine/autocrine manner. Perhaps an analogy to the possible discrepancy between LLL effects in vitro versus in vivo may be made with the medical practice of extracorporeal ozonation of blood. This practice is similar to LLL therapy given that it is used in treatment of conditions such as atherosclerosis, non-healing ulcers, and various degenerative conditions, despite no clear mechanistic understanding [59-61]. In vitro studies have demonstrated that ozone is a potent oxidant and inducer of cell apoptosis and inflammatory signaling [62-64]. In contrast, in vivo systemic changes subsequent to administration of ozone or ozonized blood in animal models and patients are quite the opposite. Numerous investigators have published enhanced anti-oxidant enzyme activity such as elevations in Mg-SOD and glutathione-peroxidase levels, as well as diminishment of inflammation-associated pathology [65-68]. Regardless of the complexity of in vivo situations, the fact that reproducible, in vitro experiments, demonstrate a biological effect provided support for us that there is some basis for LLL and it is not strictly an area of phenomenology.

In one embodiment of the invention, light is applied to the penile area for stimulation of stem cell/progenitor homing, or direct activation. Accordingly, some of the studies are described as a reference for one of skill in the art in applying light to in vivo systems. As early as 1983, Surinchak et al reported in a rat skin incision healing model that wounds exposed HeNe radiation of fluency 2.2 J/cm(2) for 3 min twice daily for 14 days demonstrated a 55% increase in breaking strength over control rats. Interestingly, higher doses yielded poorer healing [69]. This application of laser light was performed directly on shaved skin. In a contradictory experiment, it was reported that rats irradiated for 12 days with four levels of laser light (0.0, 0.47, 0.93, and 1.73 J/cm(2)) a possible strengthening of wounds tension was observed at the highest levels of irradiation (1.73 J/cm(2)), however it did not reach significance when analyzed by resampling statistics [70]. In another wound-healing study Ghamsari et al reported accelerated healing in the cranial surface of teats in dairy cows by administration of HeNe irradiation at 3.64 J/cm2 dose of low-level laser, using a helium-neon system with an output of 8.5 mW, continuous wave [71]. Collagen fibers in LLLT groups were denser, thicker, better arranged and more continuous with existing collagen fibers than those in non-LLLT groups. The mean tensile strength was significantly greater in LLLT groups than in non-LLLT groups [72]. In the random skin flap model, the use of He—Ne laser irradiation with 3 J/cm(2) energy density immediately after the surgery and for the four subsequent days was evaluated in 4 experimental groups: Group 1 (control) sham irradiation with He—Ne laser; Group 2 irradiation by punctual contact technique on the skin flap surface; Group 3 laser irradiation surrounding the skin flap; and Group 4 laser irradiation both on the skin flap surface and around it. The percentage of necrotic area of the four groups was determined on day 7-post injury. The control group had an average necrotic area of 48.86%; the group irradiated on the skin flap surface alone had 38.67%; the group irradiated around the skin flap had 35.34%; and the group irradiated one the skin flap surface and around it had 22.61%. All experimental groups reached statistically significant values when compared to control [73]. Quite striking results were obtained in an alloxan-induced diabetes wound healing model in which a circular 4 cm(2) excisional wound was created on the dorsum of the diabetic rats. Treatment with HeNe irradiation at 4.8 J/cm(2) was performed 5 days a week until the wound healed completely and compared to sham irradiated animals. The laser-treated group healed on average by the 18th day whereas, the control group healed on average by the 59th day [74]. In addition to mechanically-induced wounds, beneficial effects of LLL have been obtained in burn-wounds in which deep second-degree burn wounds were induced in rats and the effects of daily HeNe irradiation at 1.2 and 2.4 J/cm(2) were assessed in comparison to 0.2% nitrofurazone cream. The number of macrophages at day 16, and the depth of new epidermis at day 30, was significantly less in the laser treated groups in comparison with control and nitrofurazone treated groups. Additionally, infections with S. epidermidis and S. aureus were significantly reduced [75].

While numerous studies have examined dermatological applications of LLL, which may conceptually be easier to perform due to ability to topically apply light, extensive investigation has also been made in the area of orthopedic applications. Healing acceleration has been observed in regeneration of the rat mid-cortical diaphysis of the tibiae, which is a model of post-injury bone healing. A small hole was surgically made with a dentistry burr in the tibia and the injured area and LLL was administered over a 7 or 14 day course transcutaneously starting 24 h from surgery. Incident energy density dosages of 31.5 and 94.5 Jcm(−2) were applied during the period of the tibia wound healing. Increased angiogenesis was observed after 7 days irradiation at an energy density of 94.5 Jcm(−2), but significantly decreased the number of vessels in the 14-day irradiated tibiae, independent of the dosage [76]. In an osteoarthritis model treatment with HeNe resulted in augmentation of heat shock proteins and pathohistological improvement of arthritic cartilage [77]. The possibility that a type of preconditioning response is occurring, which would involve induction of genes such as hemoxygenase-1 [78], remains to be investigated. Effects of LLL therapy on articular cartilage were confirmed by another group. The experiment consisted of 42 young Wistar rats whose hind limbs were operated on in order to immobilize the knee joint. One week after operation they were assigned to three groups; irradiance 3.9 W/cm2, 5.8 W/cm2, and sham treatment. After 6 times of treatment for another 2 weeks significantpreservation of articular cartilage stiffness with 3.9 and 5.8 W/cm2 therapy was observed [79].

Muscle regeneration by LLL was demonstrated in a rat model of disuse atrophy in which eight-week-old rats were subjected to hindlimb suspension for 2 weeks, after which they were released and recovered. During the recovery period, rats underwent daily LLL irradiation (Ga—Al—As laser; 830 nm; 60 mW; total, 180 s) to the right gastrocnemius muscle through the skin. After 2-weeks the number of capillaries and fibroblast growth factor levels exhibited significant elevation relative to those of the LLL-untreated muscles. LLL treatment induced proliferation in satellite cells as detected by BRdU [80].

Other animal studies of LLL have demonstrated effects in areas that appear unrelated such as suppression of snake venom induced muscle death [81], decreasing histamine-induced vasospasms [82], inhibition of post-injury restenosis [83], and immune stimulation by thymic irradiation [84].

The clinical translation of light and LLL in exposure to penile areas is guided by prior experiences which are incorporated by reference. Growth factor secretion by LLL and its apparent regenerative activities have stimulated studies in radiation-induced mucositis. A 30 patient randomized trial of carcinoma patients treated by radiotherapy alone (65 Gy at a rate of 2 Gy/fraction, 5 fractions per week) without prior surgery or concomitant chemotherapy suffering from radiation-induced mucositis was performed using a HeNe 60 mW laser. Grade 3 mucositis occured with a frequency of 35.2% in controls and at 7.6% of treated patients. Furthermore, a decrease in “severe pain” (grade 3) was observed in that 23.8% in the control group experienced this level of pain, as compared to 1.9% in the treatment group. [85]. A subsequent study reported similar effects [86].

Healing ability of lasers was also observed in a study of patients with gingival flap incisions. Fifty-eight extraction patients had one of two gingival flap incisions lased with a 1.4 mw helium-neon (670 nm) at 0.34 J/cm(2). Healing rates were evaluated clinically and photographically. Sixty-nine percent of the irradiated incisions healed faster than the control incisions. No significant difference in healing was noted when patients were compared by age, gender, race, and anatomic location of the incision [87]. Another study evaluating healing effects of LLL in dental practice examined 48 patients subjected to surgical removal of their lower third molars. Treated patients were administered Ga—Al—As diode generated 808 nm at a dose of 12 J. The study demonstrated that extraoral LLLT is more effective than intraoral LLLT, which was more effective than control for the reduction of postoperative trismus and swelling after extraction of the lower third molar [88].

Given the predominance of data supporting fibroblast proliferative ability and animal wound healing effects of LLL therapy, a clinical trial was performed on healing of ulcers. In a double-blinded fashion 23 diabetic leg ulcers from 14 patients were divided into two groups. Phototherapy was applied (<1.0 J cm(−2)) twice per week, using a Dynatron Solaris 705(R) LED device that concurrently emits 660 and 890 nm energies. At days 15, 30, 45, 60, 75, and 90 dmean ulcer granulation and healing rates were significantly higher for the treatment group as compared to control. By day 90, 58.3% of the ulcers in the LLL treated group were fully healed and 75% achieved 90-100% healing. In the placebo group only one ulcer healed fully [58].

As previously mentioned, LLL appears to have some angiogenic activity. One of the major problems in coronary artery disease is lack of collateralization. In a 39 patient study advanced CAD, two sessions of irradiation of low-energy laser light on skin in the chest area from helium-neon B1 lasers. The time of irradiation was 15 minutes while operations were performed 6 days a week for one month. Reduction in Canadian Cardiology Society (CCS) score, increased exercise capacity and time, less frequent angina symptoms during the treadmill test, longer distance of 6-minute walk test and a trend towards less frequent 1 mm ST depression lasting 1 min during Holter recordings was noted after therapy [89].

Perhaps one of the largest clinical trials with LLL was the NEST trial performed by Photothera. In this double blind trial 660 stroke patients were recruited and randomized: 331 received LLL and 327 received sham. No prespecified test achieved significance, but a post hoc analysis of patients with a baseline National Institutes of Health Stroke Scale score of <16 showed a favorable outcome at 90 days on the primary end point (P<0.044) [12].

### Example 1

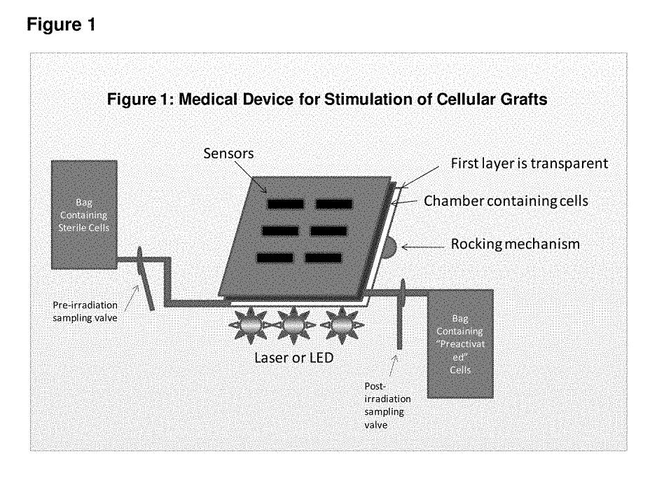

The preactivation of stem cells before administration in erectile dysfunction patients may be performed with a medical device comprised of a closed system. Said device contains an inlet that connects in a closed manner to a cryobag using connection systems available in the art such as Leuer Lock. Access to cells pre and post-irradiation is provided using valves that allow for sample collection. This is an important part of quality control for the cells. Classical parameters of interest include cell viability and morphology. Said device further consists of a surface area of sufficient size so that cells from the cryobag or other sterile source can be exposed to irradiation in a uniform or semi-uniform manner. Said device may further possess a rocking mechanism to ensure cell dispersion. Under the surface area a low level laser is provided that administers the desired frequency and intensity of laser irradiation to said cells (FIG. 1). Said device may be useful for other cellular grafts besides bone marrow cells. For example, the cells used may be islets for treatment of diabetes. In the case of islets parameters of preactivation may be different than ones used for hematopoietic stem cells. In the case of islets important considerations include the induction of the anti-apoptotic gene bcl-2, stimulation of angiogenic factors such as VEGF and IGF-1, as well as stimulation of proliferation. One of skill in the art will use the teachings of the current invention to screen for wavelengths and energy densities capable of accomplishing these goals.

### REFERENCES

1. Gratwohl, A. and H. Baldomero, Trends of hematopoietic stem cell transplantation in the third millennium. Curr Opin Hematol, 2009. 16(6): p. 420-6.
2. Ryan, E. A., et al., Clinical outcomes and insulin secretion after islet transplantation with the Edmonton protocol. Diabetes, 2001. 50(4): p. 710-9.
3. Sakata, N., et al., Bone marrow cells produce nerve growth factor and promote angiogenesis around transplanted islets. World J Gastroenterol. 16(10): p. 1215-20.
4. Sakata, N., et al., Hyperbaric oxygen therapy improves early posttransplant islet function. Pediatr Diabetes.
5. Park, K. S., et al., Trophic molecules derived from human mesenchymal stem cells enhance survival, function, and angiogenesis of isolated islets after transplantation. Transplantation. 89(5): p. 509-17.
6. Gregoraszczuk, E., J. W. Dobrowolski, and J. Galas, Effect of low intensity laser beam on steroid dehydrogenase activity and steroid hormone production in cultured porcine granulosa cells. Folia Histochem Cytochem (Krakow), 1983. 21(2): p. 87-92.
7. Yu, H. S., et al., Low-energy helium-neon laser irradiation stimulates interleukin-1 alpha and interleukin-8 release from cultured human keratinocytes. J Invest Dermatol, 1996. 107(4): p. 593-6.
8. Zhang, H., et al., Low Level Laser Irradiation Precondition to Create Friendly Milieu of Infarcted Myocardium and Enhance Early Survival of Transplanted Bone Marrow Cells. J Cell Mol Med, 2009.
9. Hou, J. F., et al., In vitro effects of low-level laser irradiation for bone marrow mesenchymal stem cells: proliferation, growth factors secretion and myogenic differentiation. Lasers Surg Med, 2008. 40(10): p. 726-33.
10. Tuby, H., L. Maltz, and U. Oron, Low-level laser irradiation (LLLI) promotes proliferation of mesenchymal and cardiac stem cells in culture. Lasers Surg Med, 2007. 39(4): p. 373-8.
11. Tuby, H., L. Maltz, and U. Oron, Implantation of low-level laser irradiated mesenchymal stem cells into the infarcted rat heart is associated with reduction in infarct size and enhanced angiogenesis. Photomed Laser Surg, 2009. 27(2): p. 227-33.
12. Zivin, J. A., et al., Effectiveness and safety of transcranial laser therapy for acute ischemic stroke. Stroke, 2009. 40(4): p. 1359-64.
13. Maiman T. H. Stimulated optical radiation in Ruby. Nature 187:493.
14. Roy, D., Ablative facial resurfacing. Dermatol Clin, 2005. 23(3): p. 549-59, viii.
15. Brown, M. C., An evidence-based approach to patient selection for laser vision correction. J Refract Surg, 2009. 25(7 Suppl): p. S661-7.
16. Brancaleon, L. and H. Moseley, Laser and non-laser light sources for photodynamic therapy. Lasers Med Sci, 2002. 17(3): p. 173-86.
17. Mester, E. S., B., and Tota, J. G. (1967). “Effect of laser on hair growth of mice”. Kiserl Orvostud 19: 628-631.
18. Mester, E., et al., The effect of laser irradiation on the regeneration of muscle fibers (preliminary report). Z Exp Chir, 1975. 8(4): p. 258-62.
19. Posten, W., et al., Low-level laser therapy for wound healing: mechanism and efficacy. Dermatol Surg, 2005. 31(3): p. 334-40.
20. Vladimirov, Y. A., A. N. Osipov, and G. I. Klebanov, Photobiological principles of therapeutic applications of laser radiation. Biochemistry (Mosc), 2004. 69(1): p. 81-90.
21. Hu, W. P., et al., Helium-neon laser irradiation stimulates cell proliferation through photostimulatory effects in mitochondria. J Invest Dermatol, 2007. 127(8): p. 2048-57.
22. Moriyama, Y., et al., In vivo effects of low level laser therapy on inducible nitric oxide synthase. Lasers Surg Med, 2009. 41(3): p. 227-31.
23. Samoilova, K. A., et al., Role of nitric oxide in the visible light-induced rapid increase of human skin microcirculation at the local and systemic levels: II. healthy volunteers. Photomed Laser Surg, 2008. 26(5): p. 443-9.
24. Yamaura, M., et al., Low level light effects on inflammatory cytokine production by rheumatoid arthritis synoviocytes. Lasers Surg Med, 2009. 41(4): p. 282-90.
25. Shiba, H., et al., Neodymium-doped yttrium-aluminium-garnet laser irradiation abolishes the increase in interleukin-6 levels caused by peptidoglycan through the p38 mitogen-activated protein kinase pathway in human pulp cells. J Endod, 2009. 35(3): p. 373-6.
26. Mafra de Lima, F., et al., Low level laser therapy (LLLT): attenuation of cholinergic hyperreactivity, beta(2)-adrenergic hyporesponsiveness and TNF-alpha mRNA expression in rat bronchi segments in E. coli lipopolysaccharide-induced airway inflammation by a NF-kappaB dependent mechanism. Lasers Surg Med, 2009. 41(1): p. 68-74.
27. Aimbire, F., et al., Low level laser therapy (LLLT) decreases pulmonary microvascular leakage, neutrophil influx and IL-1 beta levels in airway and lung from rat subjected to LPS-induced inflammation. Inflammation, 2008. 31(3): p. 189-97.
28. Safavi, S. M., et al., Effects of low-level He-Ne laser irradiation on the gene expression of IL-1beta, TNF-alpha, IFN-gamma, TGF-beta, bFGF, and PDGF in rat's gingiva. Lasers Med Sci, 2008. 23(3): p. 331-5.
29. Saygun, I., et al., Effects of laser irradiation on the release of basic fibroblast growth factor (bFGF), insulin like growth factor-1 (IGF-1), and receptor of IGF-1 (IGFBP3) from gingival fibroblasts. Lasers Med Sci, 2008. 23(2): p. 211-5.
30. Schwartz, F., et al., Effect of helium/neon laser irradiation on nerve growth factor synthesis and secretion in skeletal muscle cultures. J Photochem Photobiol B, 2002. 66(3): p. 195-200.
31. Yu, W., J. O. Naim, and R. J. Lanzafame, The effect of laser irradiation on the release of bFGF from 3T3 fibroblasts. Photochem Photobiol, 1994. 59(2): p. 167-70.
32. Zungu, I. L., D. Hawkins Evans, and H. Abrahamse, Mitochondrial responses of normal and injured human skin fibroblasts following low level laser irradiation—an in vitro study. Photochem Photobiol, 2009. 85(4): p. 987-96.
33. Wu, S., et al., High fluence low-power laser irradiation induces mitochondrial permeability transition mediated by reactive oxygen species. J Cell Physiol, 2009. 218(3): p. 603-11.
34. Lan, C. C., et al., Low-energy helium-neon laser induces melanocyte proliferation via interaction with type IV collagen: visible light as a therapeutic option for vitiligo. Br J Dermatol, 2009. 161(2): p. 273-80.
35. Karu, T., Photobiology of low-power laser effects. Health Phys, 1989. 56(5): p. 691-704.
36. Tiphlova, O. and T. Karu, Role of primary photoacceptors in low-power laser effects: action of He-Ne laser radiation on bacteriophage T4-Escherichia coli interaction. Lasers Surg Med, 1989. 9(1): p. 67-9.
37. Karu, T. I., Mitochondrial signaling in mammalian cells activated by red and near-IR radiation. Photochem Photobiol, 2008. 84(5): p. 1091-9.
38. Zhang, L., et al., Low-power laser irradiation inhibiting Abeta25-35-induced PC12 cell apoptosis via PKC activation. Cell Physiol Biochem, 2008. 22(1-4): p. 215-22.
39. Aimbire, F., et al., Low-level laser therapy decreases levels of lung neutrophils anti-apoptotic factors by a NF-kappaB dependent mechanism. Int Immunopharmacol, 2008. 8(4): p. 603-5.
40. Lipovsky, A., Y. Nitzan, and R. Lubart, A possible mechanism for visible light-induced wound healing. Lasers Surg Med, 2008. 40(7): p. 509-14.
41. Ignatieva, N., et al., Effects of laser irradiation on collagen organization in chemically induced degenerative annulus fibrosus of lumbar intervertebral disc. Lasers Surg Med, 2008. 40(6): p. 422-32.
42. Silveira, L. B., et al., Investigation of mast cells in human gingiva following low-intensity laser irradiation. Photomed Laser Surg, 2008. 26(4): p. 315-21.
43. Coombe, A. R., et al., The effects of low level laser irradiation on osteoblastic cells. Clin Orthod Res, 2001. 4(1): p. 3-14.
44. Mvula, B., et al., The effect of low level laser irradiation on adult human adipose derived stem cells. Lasers Med Sci, 2008. 23(3): p. 277-82.
45. Mvula, B., T. J. Moore, and H. Abrahamse, Effect of low-level laser irradiation and epidermal growth factor on adult human adipose-derived stem cells. Lasers Med Sci. 25(1): p. 33-9.
46. Hawkins, D. H. and H. Abrahamse, The role of laser fluence in cell viability, proliferation, and membrane integrity of wounded human skin fibroblasts following helium-neon laser irradiation. Lasers Surg Med, 2006. 38(1): p. 74-83.
47. Houreld, N. and H. Abrahamse, In vitro exposure of wounded diabetic fibroblast cells to a helium-neon laser at 5 and 16 J/cm2. Photomed Laser Surg, 2007. 25(2): p. 78-84.
48. Smol'yaninova, N. K., et al., Effects of He-Ne laser irradiation on chromatin properties and synthesis of nucleic acids in human peripheral blood lymphocytes. Biomed Sci, 1991. 2(2): p. 121-6.
49. Funk, J. O., et al., Helium-neon laser irradiation induces effects on cytokine production at the protein and the mRNA level. Exp Dermatol, 1993. 2(2): p. 75-83.
50. Funk, J. O., A. Kruse, and H. Kirchner, Cytokine production after helium-neon laser irradiation in cultures of human peripheral blood mononuclear cells. J Photochem Photobiol B, 1992. 16(3-4): p. 347-55.
51. Gulsoy, M., et al., The biological effects of 632.8-nm low energy He-Ne laser on peripheral blood mononuclear cells in vitro. J Photochem Photobiol B, 2006. 82(3): p. 199-202.
52. Novoselova, E. G., et al., [Effect of low-intensity laser radiation (632.8 nm) on immune cells isolated from mice]. Biofizika, 2006. 51(3): p. 509-18.
53. Dube, A., H. Bansal, and P. K. Gupta, Modulation of macrophage structure and function by low level He-Ne laser irradiation. Photochem Photobiol Sci, 2003. 2(8): p. 851-5.
54. Hemvani, N., D. S. Chitnis, and N. S. Bhagwanani, Helium-neon and nitrogen laser irradiation accelerates the phagocytic activity of human monocytes. Photomed Laser Surg, 2005. 23(6): p. 571-4.
55. Moustsen, P. A., et al., [Laser treatment of sinusitis in general practice assessed by a double-blind controlled study]. Ugeskr Laeger, 1991. 153(32): p. 2232-4.
56. Shen, X., et al., Effect of combined laser acupuncture on knee osteoarthritis: a pilot study. Lasers Med Sci, 2009. 24(2): p. 129-36.
57. Ekim, A., et al., Effect of low level laser therapy in rheumatoid arthritis patients with carpal tunnel syndrome. Swiss Med Wkly, 2007. 137(23-24): p. 347-52.
58. Minatel, D. G., et al., Phototherapy promotes healing of chronic diabetic leg ulcers that failed to respond to other therapies. Lasers Surg Med, 2009. 41(6): p. 433-41.
59. Bocci, V., V. Travagli, and I. Zanardi, May oxygen-ozone therapy improves cardiovascular disorders? Cardiovasc Hematol Disord Drug Targets, 2009. 9(2): p. 78-85.
60. Bocci, V., et al., The ozone paradox: ozone is a strong oxidant as well as a medical drug. Med Res Rev, 2009. 29(4): p. 646-82.
61. Re, L., et al., Ozone therapy: clinical and basic evidence of its therapeutic potential. Arch Med Res, 2008. 39(1): p. 17-26.
62. Damera, G., et al., Ozone modulates IL-6 secretion in human airway epithelial and smooth muscle cells. Am J Physiol Lung Cell Mol Physiol, 2009. 296(4): p. L674-83.
63. Manzer, R., et al., Ozone exposure of macrophages induces an alveolar epithelial chemokine response through IL-1alpha. Am J Respir Cell Mol Biol, 2008. 38(3): p. 318-23.
64. McDonald, R. J. and J. Usachencko, Neutrophils injure bronchial epithelium after ozone exposure. Inflammation, 1999. 23(1): p. 63-73.
65. Rodriguez, Z. Z., et al., Preconditioning with ozone/oxygen mixture induces reversion of some indicators of oxidative stress and prevents organic damage in rats with fecal peritonitis. Inflamm Res, 2009.
66. Zamora, Z. B., et al., Effects of ozone oxidative preconditioning on TNF-alpha release and antioxidant-prooxidant intracellular balance in mice during endotoxic shock. Mediators Inflamm, 2005. 2005(1): p. 16-22.
67. Borrego, A., et al., Protection by ozone preconditioning is mediated by the antioxidant system in cisplatin-induced nephrotoxicity in rats. Mediators Inflamm, 2004. 13(1): p. 13-9.
68. Martinez-Sanchez, G., et al., Therapeutic efficacy of ozone in patients with diabetic foot. Eur J Pharmacol, 2005. 523(1-3): p. 151-61.
69. Surinchak, J. S., et al., Effects of low-level energy lasers on the healing of full-thickness skin defects. Lasers Surg Med, 1983. 2(3): p. 267-74.
70. Broadley, C., et al., Low-energy helium-neon laser irradiation and the tensile strength of incisional wounds in the rat. Wound Repair Regen, 1995. 3(4): p. 512-7.
71. Ghamsari, S. M., et al., Histopathological effect of low-level laser therapy on sutured wounds of the teat in dairy cattle. Vet Q, 1996. 18(1): p. 17-21.
72. Ghamsari, S. M., et al., Evaluation of low level laser therapy on primary healing of experimentally induced full thickness teat wounds in dairy cattle. Vet Surg, 1997. 26(2): p. 114-20.
73. Pinfildi, C. E., et al., Helium-neon laser in viability of random skin flap in rats. Lasers Surg Med, 2005. 37(1): p. 74-7.
74. Maiya, G. A., P. Kumar, and L. Rao, Effect of low intensity helium-neon (He-Ne) laser irradiation on diabetic wound healing dynamics. Photomed Laser Surg, 2005. 23(2): p. 187-90.
75. Bayat, M., et al., Effect of low-level laser therapy on the healing of second-degree burns in rats: a histological and microbiological study. J Photochem Photobiol B, 2005. 78(2): p. 171-7.
76. Garavello, I., V. Baranauskas, and M. A. da Cruz-Hofling, The effects of low laser irradiation on angiogenesis in injured rat tibiae. Histol Histopathol, 2004. 19(1): p. 43-8.
77. Lin, Y. S., et al., Effects of helium-neon laser on levels of stress protein and arthritic histopathology in experimental osteoarthritis. Am J Phys Med Rehabil, 2004. 83(10): p. 758-65.
78. Jamieson, R. W. and P. J. Friend, Organ reperfusion and preservation. Front Biosci, 2008. 13: p. 221-35.
79. Akai, M., et al., Laser's effect on bone and cartilage change induced by joint immobilization: an experiment with animal model. Lasers Surg Med, 1997. 21(5): p. 480-4.
80. Nakano, J., et al., Low-level laser irradiation promotes the recovery of atrophied gastrocnemius skeletal muscle in rats. Exp Physiol, 2009. 94(9): p. 1005-15.
81. Doin-Silva, R., et al., The ability of low level laser therapy to prevent muscle tissue damage induced by snake venom. Photochem Photobiol, 2009. 85(1): p. 63-9.
82. Gal, D., et al., Percutaneous delivery of low-level laser energy reverses histamine-induced spasm in atherosclerotic Yucatan microswine. Circulation, 1992. 85(2): p. 756-68.
83. Kipshidze, N., et al., Photoremodeling of arterial wall reduces restenosis after balloon angioplasty in an atherosclerotic rabbit model. J Am Coll Cardiol, 1998. 31(5): p. 1152-7.
84. Novoselova, E. G., et al., Effects of low-power laser radiation on mice immunity. Photodermatol Photoimmunol Photomed, 2006. 22(1): p. 33-8.
85. Bensadoun, R. J., et al., Low-energy He/Ne laser in the prevention of radiation-induced mucositis. A multicenter phase III randomized study in patients with head and neck cancer. Support Care Cancer, 1999. 7(4): p. 244-52.
86. Arun Maiya, G., M. S. Sagar, and D. Fernandes, Effect of low level helium-neon (He-Ne) laser therapy in the prevention & treatment of radiation induced mucositis in head & neck cancer patients. Indian J Med Res, 2006. 124(4): p. 399-402.
87. Neiburger, E. J., Rapid healing of gingival incisions by the helium-neon diode laser. J Mass Dent Soc, 1999. 48(1): p. 8-13, 40.
88. Aras, M. H. and M. Gungormus, Placebo-controlled randomized clinical trial of the effect two different low-level laser therapies (LLLT)-intraoral and extraoral-on trismus and facial swelling following surgical extraction of the lower third molar. Lasers Med Sci, 2009.
89. Zycinski, P., et al., Laser biostimulation in end-stage multivessel coronary artery disease—a preliminary observational study. Kardiol Pol, 2007. 65(1): p. 13-21; discussion 22-3.

1. A method of increasing efficacy of a cell for use in transplantation by pretreatment of said cell prior to transplantation with a laser irradiation of at least one wavelength, said wavelength(s) in a range between about 400 nanometers and about 1070 nanometers administered for a total energy of 100 W/cm² to approximately 10 W/cm²

2. The method of claim 1, wherein said transplanted cell is a bone marrow mononuclear cell population.

3. The method of claim 2, wherein said bone marrow mononuclear cell population is purified for CD133 positive cells.

4. The method of claim 1, wherein said cell population is adipose stromal vascular fraction cells.

5. The method of claim 4, wherein said adipose stromal vascular fraction cells are used as a source of mesenchymal stem cells.

6. The method of claim 4, wherein said mesenchymal stem cells are autologous or allogeneic.

7. The method of claim 2, wherein said bone marrow cells are isolated based on expression of CD34.

8. The method of claim 2, wherein said bone marrow cells are isolated based on expression of aldehyde dehydrogenase activity.

9. The method of claim 1, wherein said cells are mobilized peripheral blood stem cells comprised of a heterogeneous leukopheresis product extracted from a donor after mobilization.

10. The method of claim 9, wherein said mobilized peripheral blood stem cells are comprised of an isolated CD34 cell derived from heterogeneous leukopheresis product extracted from a donor after mobilization.

11. The method of claim 1, wherein said cells are cord blood derived stem cells.

12. The method of claim 11, wherein said cord blood derived stem cells comprise a population selected for expression of CD34.

13. The method of claim 11, wherein said cord blood derived stem cells comprise a population selected for expression of CD133.

14. The method of claim 1, wherein said pretreated cells are transplanted into a patient having suboptimal corpus cavernosal tissue in an amount sufficient to treat said corpus cavernosal tissue.

15. A method of treating suboptimal function of corpus cavernosal tissue comprising the steps of: a) selecting a patient whose corpus cavernosal tissue is functioning suboptimally; b) providing said patient a mobilizing agent or therapy sufficient to increase circulating regenerative cells; and c) providing said patient a chemoattractant to selectively induce homing of said regenerative cells to said corpus cavernosal tissue.
